(19) United States
(12) Patent Application Publication
(10) Pub. No.: US 2018/0098799 A1
(43) Pub. Date: Apr. 12, 2018

(54) CABLE BONE SCREW, TOOLS, AND METHODS

(71) Applicant: MATTHEW SONGER, MARQUETTE, MI (US)

(72) Inventor: MATTHEW SONGER, MARQUETTE, MI (US)

(21) Appl. No.: 15/728,491

(22) Filed: Oct. 9, 2017

Related U.S. Application Data
(60) Provisional application No. 62/405,876, filed on Oct. 8, 2016.

(51) Int. Cl. A61B 17/70; A61B 17/86

(52) U.S. Cl. CPC A61B 17/7083; A61B 17/7082; A61B 17/8685; A61B 17/8605

(57) ABSTRACT

A cable bone screw implant and insertion instrument assembly comprising an implantable bone screw having an enlarged head portion. An enlarged head comprises a lock aperture and an insert aperture and holds a deformable stress tube for housing a surgical cable. A lock screw resides in the lock aperture. A control mate on the enlarged head engages with an inserter instrument which comprises an arm rod portion having a driver cannula for housing a locking rod instrument. An end of the elongate rod body is enlarged having a first and second deflector arm. An interlock engages a cable bone screw implant portion. A compression taper on the arm rod portion compresses deflector arms securing the implant. An outer tube housing engages the deflector arms. Sliding the outer tube housing locks or releases the implantable bone screw. A surgical cable is tensioned between two cable bone screw implants for fixation.

This application claims priority to Provisional Patent Application No. 62/405,876 filed Oct. 8, 2016, the entire disclosure of which is hereby incorporated by reference and relied upon.

### BACKGROUND OF THE INVENTION

Field of the Invention. The invention relates generally to orthopedic implants and orthopedic tools and methods, and more particularly to orthopedic implants comprising a cable bone screw implant, an inserter, and methods of use.

Description of Related Art. Kilpela et al. discloses a Surgical Cable Screw Connector in U.S. Pat. No. 5,702,399. The device utilizes a bone screw with a typical threaded shaft. A hexagonal screw head sits at the top of the shaft and includes a cable bore extending through the head for receiving a surgical cable. A retention screw extends into the head and interferes with a malleable sleeve housed within the cable bore. As the retension screw is advanced, it causes the malleable sleeve to bend and collapse on the cable locking it in place. Although the Kilpela device is effective in open surgeries, the device is not effective for minimally invasive use. What is needed a cable bone screw implant and instrument system configured for use through a minimal surgical incision to support a minimally invasive approach.

### SUMMARY OF THE INVENTION

Novel cable bone screw implants, instruments, and methods are disclosed herein. According to one form of this invention, an implantable bone screw having a central axis is configured with an enlarged head.

In one form, an enlarged head is non-circular.

In one form, an enlarged head is generally square or rectangular.

In one form, an enlarged head comprises broken outer edges.

In one form, an enlarged head comprises opposed first and second radial walls and opposed third and fourth radial walls.

In one form, a first and second radial wall comprises corresponding first and second radial wall surfaces thereon.

In one form, a third and fourth radial wall comprises corresponding third and fourth radial wall surfaces thereon.

In one form, an enlarged head comprises a proximal head wall with corresponding proximal head wall surface thereon.

In one form, an enlarged head comprises one or more control mates for controlling said bone screw through a small incision.

In one form, an enlarged head comprises a first and a second control mate.

In one form, a first control mate is disposed on said first radial wall.

In one form, a second control mate is disposed on said second radial wall.

In some forms a first control mate is in the form of a groove, ridge, boss, or depression.

In some forms a second control mate is in the form of a groove, ridge, boss, or depression.

In one form, a tunnel wall extends through an enlarged head generally perpendicular to a central axis of an implantable bone screw.

In one form, a tunnel wall extends from a third radial wall to a fourth radial wall.

In one form, a tunnel wall defines an insert aperture.

In one form, an insert aperture is generally cylindrical.

In one form, a tunnel wall comprises a generally uniform first bore.

In one form, a tunnel wall comprises a first and a second aligned bore of differing diameters.

In one form, a first insert step resides between said first and second aligned bore of differing diameters.

In one form, a lock wall defines a lock aperture.

In one form, a lock aperture extends from a proximal end of an implantable bone screw.

In one form, an elongate axis of a lock aperture is generally coincident with an elongate axis of an implantable bone screw.

In one form, a tunnel wall and lock wall intersect wherein a lock aperture intersects an insert aperture.

In one form, a tunnel wall comprises a tunnel surface on said tunnel wall and a lock wall comprises a lock surface on said lock wall.

In one form, inscribed on said lock surface are first threads.

In one form, an implantable bone screw comprises a shaft portion.

In one form, a shaft portion has a shaft surface.

In one form, a shaft surface comprises bone screw threads.

In one form, bone screw threads on the shaft surface are self-tapping.

In one form, a shaft portion has a tapered tip at a distal end of said bone screw.

In one form, a shaft portion has one or more cutting flutes at a distal end.

In one form, a shaft portion comprises bone screw threads having a major diameter and a minor diameter.

In one form, a shaft portion comprises an increasing minor diameter towards an enlarged head of a bone screw.

In one form, a lock screw resides within said lock aperture.

In one form, a lock screw comprises a screw body.

In one form, a lock screw comprises an engagement face at a distal end for compressive engagement with an insert.

In one form, a lock screw comprises an advance face extending radially along a longitudinal axis of a lock screw.

In one form, a lock screw comprises advance threads disposed on said advance face.

In one form, an enlarged upper rim extends from said advance face.

In one form, an enlarged upper rim comprises a stop face for abutting against a proximal head wall of an implantable bone screw.

In one form, a lock screw comprises a near face on a proximal end of said lock screw.

In one form, a lock screw comprises a drive pocket extending from a near face along a central axis of said lock screw wherein the drive pocket is defined by drive pocket walls.

In one form, a drive pocket is non-circular.

In one form, a drive pocket comprises multiple lobes for transfer of torque to the lock screw from an instrument.

In one form, a pocket slope tapers between a near face to a drive pocket.

In some forms, an end wall defines the distal end of a drive pocket.

In some forms, locking engagement between a lock screw and a lock aperture of an implantable bone screw is defined as a bayonet connection as opposed to a threaded connection.

In one form, a stress tube resides within an insert aperture.

In one form, a stress tube comprises an inner tube surface extending along an elongate axis of said tube and defining a stress aperture extending through the inside of the stress tube.

In one form, a stress aperture is sized to pass a surgical cable therethrough.

In one form, a stress tube is deformable.

In one form, a stress tube comprises a first outer tube surface extending along an elongate axis of said tube.

In one form, a stress tube comprises a second outer tube surface extending along an elongate axis of said tube.

In one form, a first outer tube surface and a second outer tube surface have unequal diameters.

In one form, an outer tube surface comprises an outer step to transition between a first and a second outer tube surface.

In one form, a stress tube comprises an entry wall and an exit wall at opposed ends of a stress tube.

In one form, an entry wall is tapered for eased insertion of a surgical cable into a stress tube.

In one form, an entry wall is adjacent the first or second outer tube surface having the larger diameter.

In one form, implant portions of a cable bone screw implant portion, surgical cable, and surgical grommet are manufactured from one or more of; titanium alloys, stainless steel alloys, and cobalt chrome alloys. Other biocompatible materials may also be useable.

In one form, an insertion instrument and a pair of cable bone screw implant portions and complementing surgical cable and optional surgical grommet are utilized for repair of one or more of pars interarticularis defects and spondylolisthesis.

In one form, a cable bone screw implant portion and complementing surgical cable and optional surgical grommet are used as an adjunct to a rigid, posterior fixation system.

In one form, an insertion instrument comprises a handle portion, an arm rod portion, and an outer tube housing.

In one form, an outer tube housing comprises an elongate tube body.

In one form, an elongate tube body comprises a first end wall at a distal end and a second end wall at a proximal end.

In one form, an elongate tube body comprises an internal body face defining an internal chamber of an elongate tube body.

In one form, an elongate tube body comprises an external body face.

In one form, an elongate tube body comprises an internal body face and opposed receiving faces which define a knuckle space for receiving clasp ends of an arm tube portion.

In one form, opposed clearance slots extend through opposed receiving faces and an external body face of an elongate tube body for the passage of surgical cable.

In one form, an elongate tube body comprises one or more cleaning slots extending between an internal body face and an external body face.

In one form, an internal chamber of an elongate tube body is sized for sliding engagement with an arm tube portion.

In one form, an external body face comprises a textured surface at a proximal end for enhanced hand gripping by a user.

In one form, a textured surface is in the form of radial ridges encircling an external body face.

In one form, an arm rod portion comprises an elongate rod body having a driver cannula extending therethrough along a central axis of the arm rod portion.

In one form, an arm rod portion comprises a divider slot splitting a distal end of the arm rod portion forming two or more opposed deflector arms.

In one form, a driver cannula comprises a cannula face formed thereon.

In one form, an arm rod portion comprises an outer surface encircling the arm rod portion on an outer face.

In one form, an arm rod portion comprises a handle lock at a proximal end.

In one form, a handle lock is in the form of a groove, a depression, or a groove and a depression.

In one form, a deflector arm comprises a clasp end.

In one form, a distal end of an arm rod portion is enlarged to form around an enlarged head portion of an implantable bone screw.

In one form, a clasp end is enlarged.

In one form, a clasp end comprises an interlock for locking engagement with a control mate of an implantable bone screw.

In one form, an interlock is in the form of an internally facing rounded elongate ridge.

In one form, an interlock includes a head gap portion for extending partially around the head of an implantable bone screw.

In one form, a cup face resides at a proximal end of said head gap portion for opposing a proximal head wall of a cable bone screw implant when a cable bone screw implant portion is captured therein.

In one form, a compression taper extends between a clasp end and an elongate rod body.

In one form, an insertion instrument comprises a handle portion.

In one form, a handle portion comprises an elongate handle body extending along a central axis.

In one form, a handle portion comprises a handle cannula extending along a central axis defined by an inner handle surface.

In one form, a handle cannula has a diameter for receiving an outer surface of an arm rod at a distal end.

In one form, a handle cannula has a diameter sufficient for receiving a locking rod extending to a lock screw portion of an implantable bone screw.

In one form, a handle portion comprises an outer handle surface.

In one form, an outer handle surface is textured for enhanced hand gripping by a user.

In one form, a textured surface is in the form of longitudinal ridges encircling an external body face.

In one form, a handle portion comprises a lock recess for locking a handle portion to a arm rod portion.

In one form, a lock recess is configured to house a pressed or spring pin.

In one form, a lock recess is threaded for receiving a set screw.

### BRIEF DESCRIPTION OF THE SEVERAL VIEWS OF THE DRAWINGS

These and other features and advantages of the present invention will become more readily appreciated when considered in connection with the following detailed description and appended drawings, wherein:

FIG. 1 depicts an exploded perspective view of a cable bone screw implant portion according to one or more embodiments shown and described herein;

FIG. 2 depicts one side view of an implantable bone screw of a cable bone screw implant according to one or more embodiments shown and described herein;

FIG. 3 depicts another side view of the implantable bone screw implant of FIG. 2 rotated along axis-A 90 degrees according to one or more embodiments shown and described herein;

FIG. 4 depicts a top view of the implantable bone screw implant of FIG. 2 according to one or more embodiments shown and described herein;

FIG. 5 depicts a bottom view of the implantable bone screw implant of FIG. 2 according to one or more embodiments shown and described herein;

FIG. 6 depicts a bottom perspective view of a lock screw of a cable bone screw implant according to one or more embodiments shown and described herein;

FIG. 7 depicts a top perspective view of a lock screw of a cable bone screw implant according to one or more embodiments shown and described herein;

FIG. 8 depicts a top perspective view of a stress tube of a cable bone screw implant according to one or more embodiments shown and described herein;

FIG. 9 depicts a top perspective view of a lock screw of a cable bone screw implant according to one or more embodiments shown and described herein;

FIG. 10 depicts an opposing top perspective view of a stress tube of a cable bone screw implant according to one or more embodiments shown and described herein;

FIG. 11A depicts a top perspective view of a cable bone screw implant and insertion instrument assembly in a preassembled configuration according to one or more embodiments shown and described herein;

FIG. 11B depicts a top perspective view of a cable bone screw implant and insertion instrument assembly in a loaded configuration according to one or more embodiments shown and described herein;

FIG. 11C depicts a top perspective view of a cable bone screw implant and insertion instrument assembly with outer tube housing removed according to one or more embodiments shown and described herein;

FIG. 11D depicts a top perspective view of a cable bone screw implant and insertion instrument assembly with outer tube housing and arm rod portion removed illustrating a locking rod instrument engaged with a lock screw according to one or more embodiments shown and described herein;

FIG. 12 depicts a partial close-up perspective view of the engagement between a cable bone screw implant and insertion instrument assembly according to one or more embodiments shown and described herein;

FIG. 13 depicts an exploded top perspective view of a cable bone screw implant and insertion instrument assembly according to one or more embodiments shown and described herein;

FIG. 14 depicts an bottom perspective view of a handle portion of a cable bone screw implant insertion instrument according to one or more embodiments shown and described herein;

FIG. 15 depicts a top perspective view of a handle portion of a cable bone screw implant insertion instrument according to one or more embodiments shown and described herein;

FIG. 16 depicts a bottom view of a handle portion of a cable bone screw implant insertion instrument according to one or more embodiments shown and described herein;

FIG. 17 depicts a top view of a handle portion of a cable bone screw implant insertion instrument according to one or more embodiments shown and described herein;

FIG. 18 depicts a perspective view of an outer tube housing of a cable bone screw implant insertion instrument according to one or more embodiments shown and described herein;

FIG. 19 depicts a distal end perspective view of an outer tube housing of a cable bone screw implant insertion instrument according to one or more embodiments shown and described herein;

FIG. 20 depicts a cross-sectional view of an outer tube housing of a cable bone screw implant insertion instrument according to one or more embodiments shown and described herein;

FIG. 21 depicts a distal end view of an outer tube housing of a cable bone screw implant insertion instrument according to one or more embodiments shown and described herein;

FIG. 22 depicts a proximal end view of an outer tube housing of a cable bone screw implant insertion instrument according to one or more embodiments shown and described herein;

FIG. 23 depicts a proximal end view of an arm rod portion of a cable bone screw implant insertion instrument according to one or more embodiments shown and described herein;

FIG. 24 depicts a distal end view of an arm rod portion of a cable bone screw implant insertion instrument according to one or more embodiments shown and described herein;

FIG. 25 depicts a perspective view of an arm rod portion of a cable bone screw implant insertion instrument according to one or more embodiments shown and described herein;

FIG. 26 depicts a partial side view of the distal end of an arm rod portion of a cable bone screw implant insertion instrument according to one or more embodiments shown and described herein;

FIG. 27 depicts a side view of an arm rod portion of a cable bone screw implant insertion instrument according to one or more embodiments shown and described herein;

FIG. 28 depicts a perspective view of a cable bone screw implant and insertion instrument assembly (outer tube housing and arm rod portion removed) used for repair of a pars interarticularis fracture of a vertebral body according to one or more embodiments shown and described herein.

FIG. 29 depicts a posterior perspective view of a human lumbar-sacral spine model illustrating a step of insertion of a cable bone screw implant portion on the right, and illustrating a predrilled pedicle on the right according to one or more embodiments shown and described herein;

FIG. 30 depicts a posterior perspective of a lumbar vertebrae spine model with a cable bone screw implant portion implanted in a left pedicle and a second cable bone screw implant portion implanted in a right pedicle according to one or more embodiments shown and described herein;

FIG. 31 depicts a partial close up perspective view of cable bone screw implant portion implanted in a pedicle according to one or more embodiments shown and described herein. The implant is secured by an insertion instrument and a lead end of a surgical cable about to be threaded into a stress aperture;

FIG. 32 depicts a partial perspective view of a surgical cable residing in the stress apertures of a pair of bilateral cooperating implanted cable bone screw implant portions seated in pedicles of a lumbar spine according to one or more embodiments shown and described herein;

FIG. 33 depicts a partial perspective view of a surgical cable seated in the stress apertures of a pair of bilateral cable bone screw implant portions according to one or more embodiments shown and described herein. A cable grommet is used to disperse stresses and friction against the spinous process.

FIG. 34 depicts a posterior perspective view of a surgical cable extending between a pair of cable bone screw implant portions and being tensioned by a cable tensioner instrument utilized in the prior art according to one or more embodiments shown and described herein;

FIG. 35 depicts a posterior perspective view of a cable cutting instrument cutting off non-tensioned ends of a surgical cable after the fixation of the cable in each stress tube according to one or more embodiments shown and described herein;

FIG. 36 depicts a perspective view of a surgical grommet used in conjunction with the surgical cable to disperse stress over bone surfaces according to one or more embodiments shown and described herein.

### DETAILED DESCRIPTION OF SELECTED EMBODIMENTS OF THE INVENTION

Selected embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

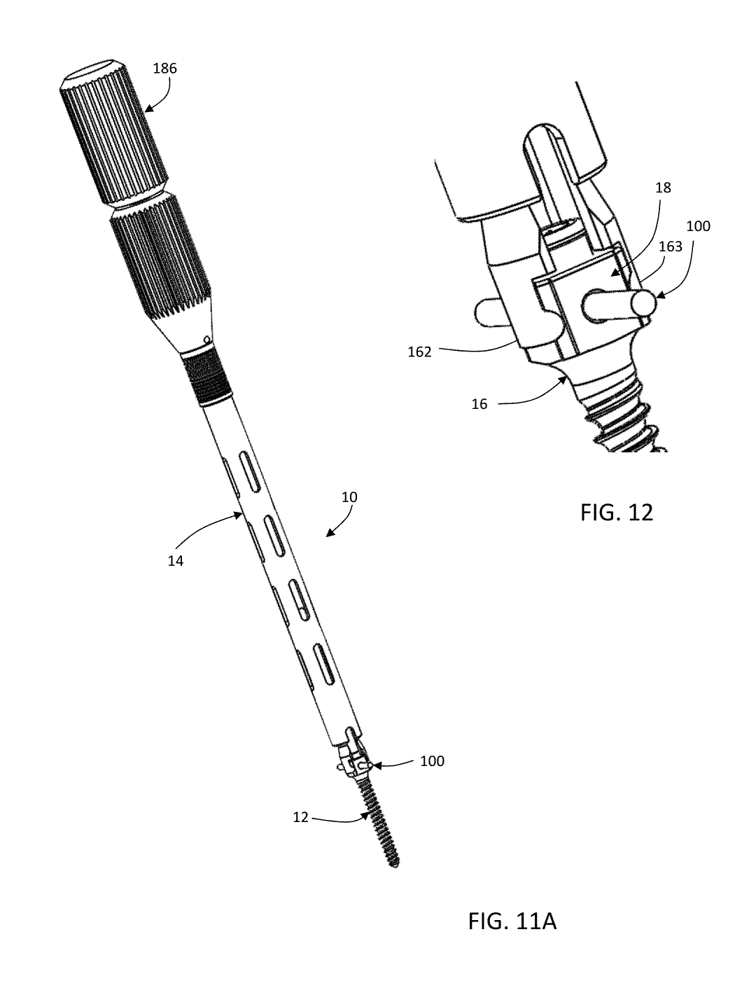

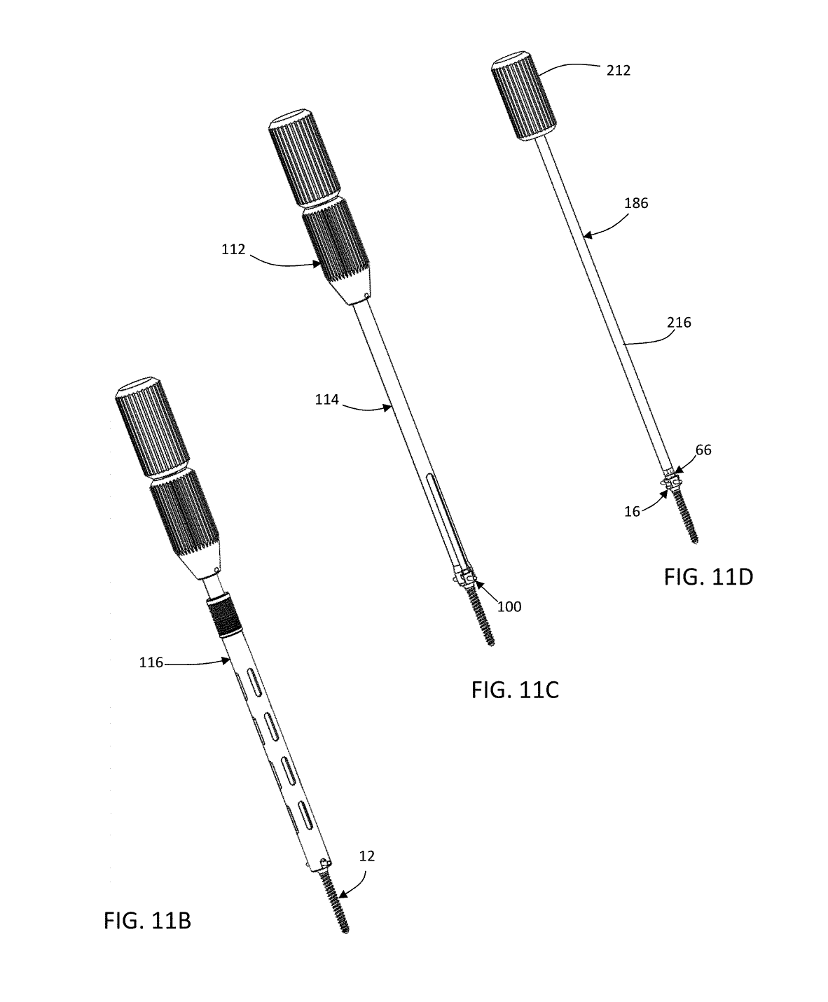

A preferred embodiment a cable bone screw implant and insertion instrument assembly 10 is illustrated in FIG. 11A in a preassembled configuration wherein an outer tube housing 116 is distracted proximally thus providing coupling and uncoupling of an implantable bone screw 16 with the instrument. A partial close-up view of the interaction between an implant and instrument is illustrated in FIG. 12. FIG. 11B depicts a cable bone screw implant and insertion instrument assembly 10 in a loaded configuration wherein an outer tube housing 116 is slid distally for locking a cable bone screw implant portion 12 to insertion instrument 14. FIG. 11C depicts a cable bone screw implant and insertion instrument assembly 10 with outer tube housing removed. FIG. 11D depicts a cable bone screw implant and insertion instrument assembly 10 with outer tube housing 116 and arm rod portion 114 removed illustrating a locking rod instrument 186 engaged with a lock screw 66 according to one or more embodiments shown and described herein.

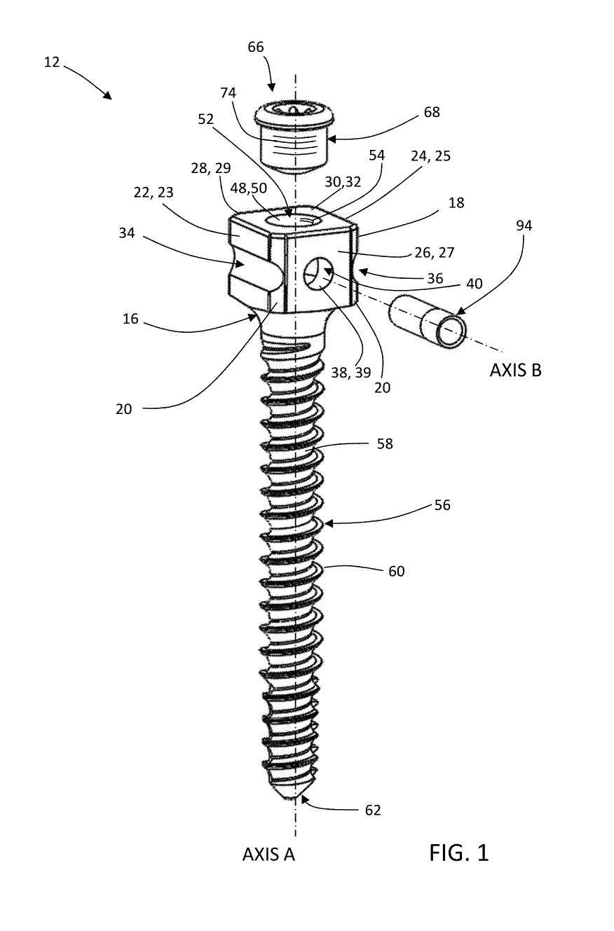

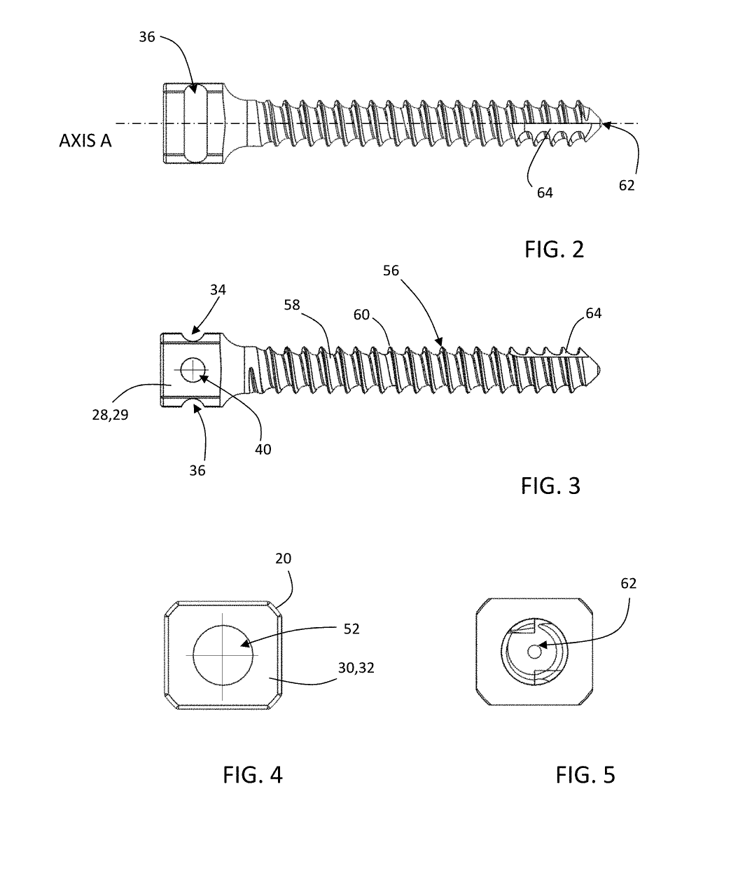

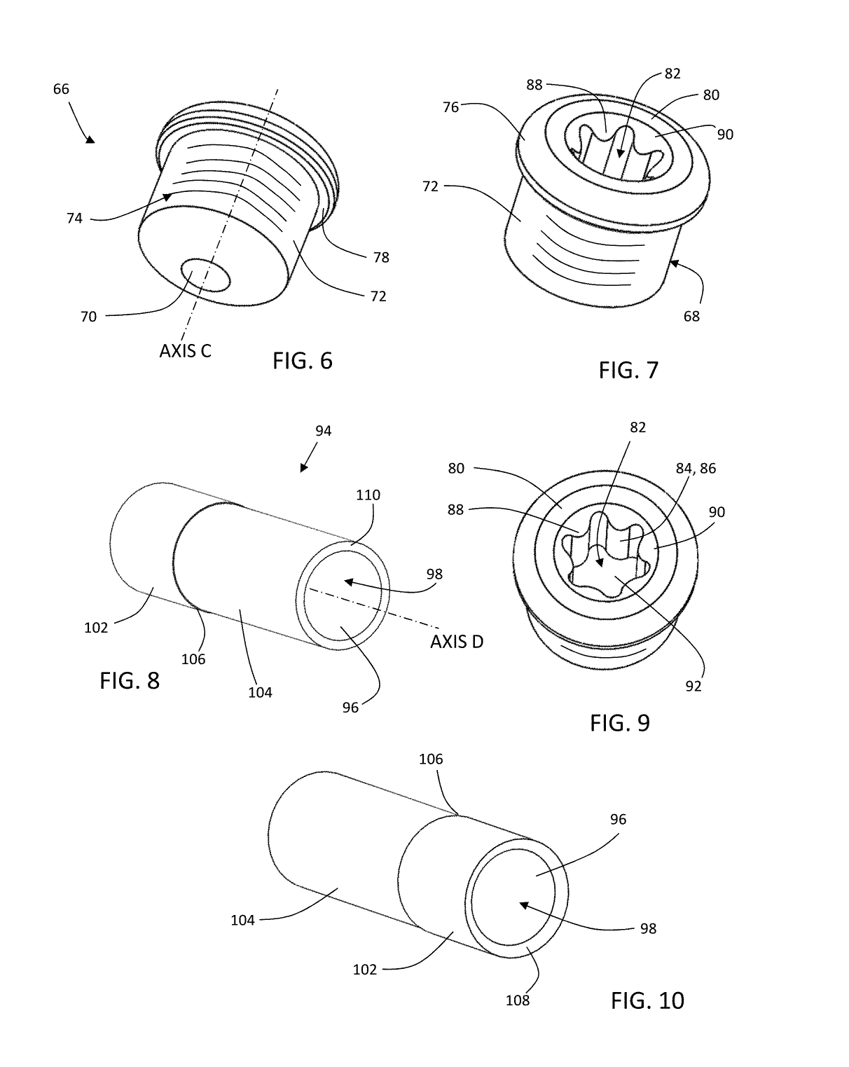

The cable bone screw implant and insertion instrument assembly 10 comprises a cable bone screw implant portion 12 and is illustrated with attached cable bone screw insertion instrument 14. Components of cable bone screw implant portion 12 are further illustrated in FIGS. 1 through 10. A cable bone screw implant portion 12 further comprises an implantable bone screw 16 portion comprising a central axis A extending along a shaft portion 56 and is configured with an enlarged head portion 18. The enlarged head 18 is preferably non-circular having a profile such as generally square or rectangular. In the embodiment of FIG. 1, the enlarged head 18 has a generally square profile. The corner edges of the square have broken outer edges 20. Enlarged head 18 further comprises opposed first radial wall 22 and second radial wall 24. In addition, enlarged head 18 comprises opposed third radial wall 26 and fourth radial wall 28 positioned generally perpendicular to said first radial wall 22 and second radial wall 24. First and second radial wall 22, 24 comprises corresponding first and second radial wall surfaces thereon 23, 25. Third and fourth radial wall 26, 28 comprises corresponding third and fourth radial wall surfaces thereon 27, 29. In this embodiment, each radial wall is generally flat and positioned generally parallel to the central axis. In addition, central axis A extending along shaft portion 56 is generally centered between opposed radial walls.

In this embodiment, an enlarged head 18 comprises a proximal head wall 30 with corresponding proximal head wall surface 32 thereon. The enlarged head 18 comprises one or more control mates 34,36 for securing and controlling said bone screw by an insertion instrument 14. As illustrated, an enlarged head 18 comprises a first control mate 34 and a second control mate 36. The first control mate 34 is disposed on first radial wall 22 whereas a second control mate 36 is disposed on said second radial wall 24. In some forms a first control mate 34 is in the form of a groove, ridge, boss, or depression and a second control mate 36 is also in the form of a groove, ridge, boss, or depression. In this embodiment, first control mate 34 and second control mate 36 are in the form of rounded channels extending generally parallel to axis B formed in the corresponding radial wall surfaces.

Tunnel wall 38 extends through enlarged head 18 along an axis B generally perpendicular to a central axis A of implantable bone screw 16. Tunnel wall 38 extends from surfaces on third radial wall 26 and fourth radial wall 28. Tunnel wall 38 defines an insert aperture 40 in which a stress tube 94 is housed. In preferred embodiments insert aperture 40 is generally cylindrical. A tunnel wall 38 comprises a generally uniform first bore 42 through enlarged head 18 however in some forms such as FIG. 1, tunnel wall 38 comprises an aligned first bore 42 and a second bore 44 of differing diameters (not shown). A first insert step 46 may reside between said first and second aligned bore 42, 44 of differing diameters.

In some forms, a lock wall 48 with corresponding lock wall surface 50 defines a lock aperture 52 extending from a proximal end of an implantable bone screw 16. An elongate axis of lock aperture 52 is generally coincident with an elongate axis-A of an implantable bone screw 16. In this embodiment, a tunnel wall 38 and lock wall 48 intersect wherein a lock aperture 52 intersects an insert aperture 40. Tunnel wall 38 comprises a tunnel wall surface 39 on the tunnel wall and a lock wall 48 comprises a lock surface 50 thereon. Inscribed on lock surface 50 are first lock threads 54 complementing advance threads 74 on lock screw 66.

Also in this embodiment of FIG. 1, implantable bone screw 16 comprises a shaft portion 56 having a shaft surface 58 comprising bone screw threads 60 thereon. Here shaft portion 56 has a tapered tip 62 at a distal end of bone screw 16. In addition, shaft portion 56 may include one or more cutting flutes 64 at a distal end as illustrated. Shaft portion 56 comprises bone screw threads 60 having a major diameter and a minor diameter. Bone screw threads 60 may be configured having an increased entry slope for eased insertion and steeper slope on proximal thread sides to increase back-out strength. As illustrated here in FIG. 1, a shaft portion 56 comprises an increasing minor diameter towards an enlarged head 18 of a bone screw 16 to increase screw strength and reduce wobble in bone.

As illustrated in FIGS. 1,6-7 and 9, lock screw 66 resides within lock aperture 52 by complementing advance threads 74 on the lock screw 66 and within lock aperture 52. Lock screw 66 comprises a lock screw body 68 and an engagement face 70 at a distal end for compressive engagement with a stress tube 94. Lock screw 66 comprises an advance face 72 extending radially along a longitudinal axis-C of a lock screw 66 and includes advance threads 74 disposed on advance face 72. At a proximal end an enlarged upper rim 76 extends from said advance face 72 and comprises a stop face 78 for abutting against a proximal head wall 30 of an implantable bone screw 16 thereby providing lock screw 66 a predetermined amount of compression on stress tube 94.

As illustrated in this embodiment, lock screw 66 comprises a near face 80 on a proximal end of said lock screw 66. Lock screw 66 further comprises drive pocket 82 extending from near face 80 along a central axis C of said lock screw 66 wherein the drive pocket 82 is defined by drive pocket faces 86 on drive pocket walls 84. As illustrated, drive pocket 82 is non-circular and comprises multiple lobes 88 to transfer torque to lock screw 66 from an insertion instrument 14. Pocket slope 90 tapers between a near face 80 to a drive pocket 82. An end wall 92 defines the distal end of a drive pocket 82.

One embodiment of a stress tube 94 is illustrated in FIG. 8, 10. Stress tube 94 resides within an insert aperture 40 of implantable bone screw 16. Stress tube 94 comprises an inner tube surface 96 extending along an elongate axis-D of said tube. Inner tube surface 96 defines a stress aperture 98 extending through the inside of the stress tube 94. Stress aperture 98 is sized to pass a surgical cable 100 therethrough. Stress tube 94 is deformable and preferably constructed of a ductile implantable metal.

Stress tube 94 comprises a first outer tube surface 102 and a second outer tube surface 104 extending along elongate axis D of tube 94. In this embodiment, first outer tube surface 102 and second outer tube surface 104 have unequal diameters but may be equal. Outer tube surfaces 102, 104 comprise an outer step 106 to transition between the first and a second outer tube surfaces. Outer step 106 assists in positioning stress tube 94 within insert aperture 40. Stress tube 94 comprises an entry wall 108 and an exit wall 110 at opposed ends of stress tube 94. Entry wall 108 is tapered for eased insertion of a surgical cable into stress tube 94. In this embodiment, entry wall 108 is adjacent the first or second outer tube surface 102, 104 having the larger diameter.

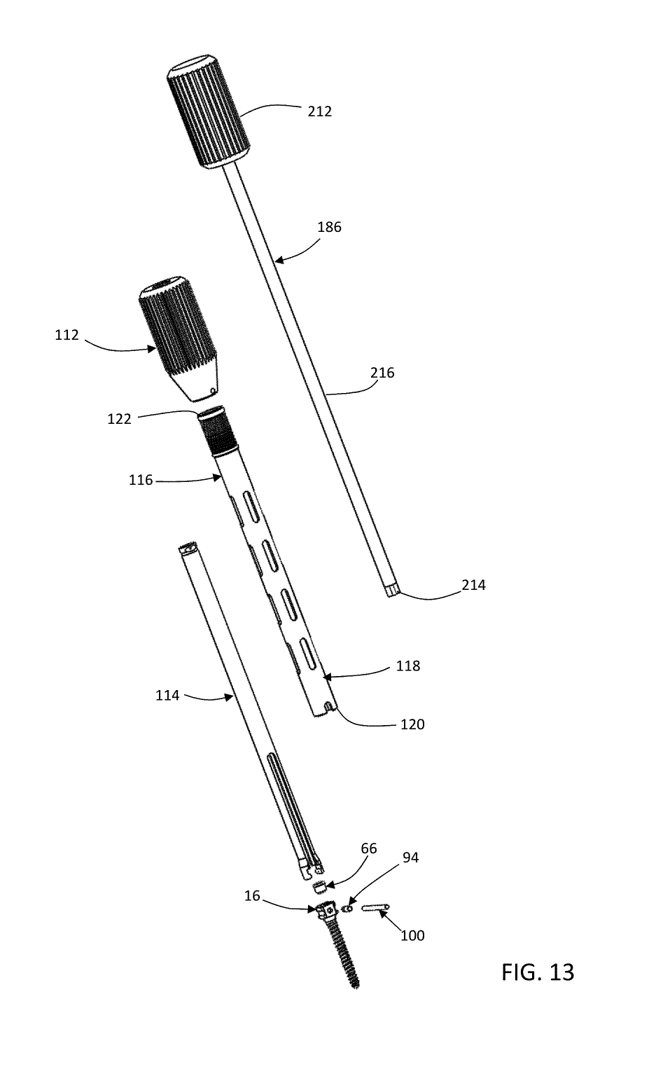

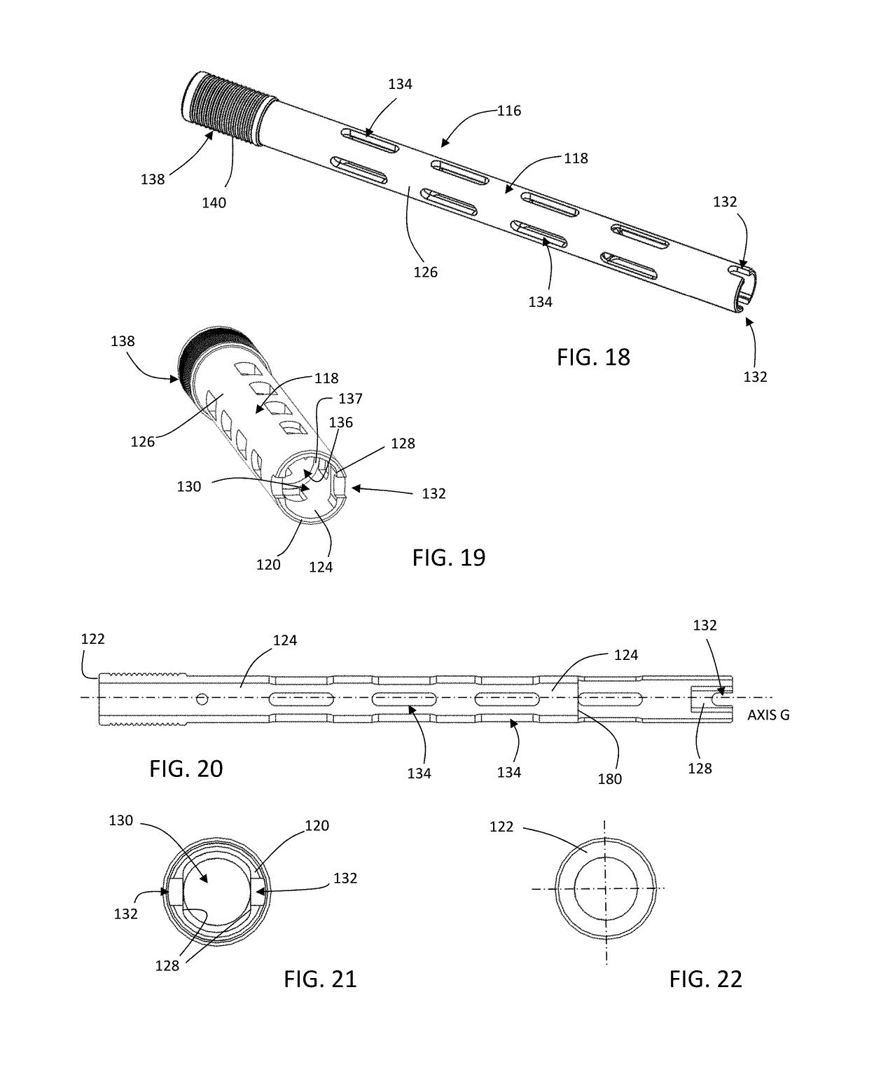

An insertion instrument 14 is illustrated in FIG. 11A and in an exploded view in FIG. 13. Insertion instrument 14 comprises a handle portion 112, an arm rod portion 114, and an outer tube housing 116. Outer tube housing 116 (FIG. 18-22) comprises an elongate tube body 118 and comprises a first end wall 120 at a distal end and a second end wall 122 at a proximal end. Further, elongate tube body 118 comprises an internal body face 124 defining an internal chamber 136 of an elongate tube body 118. Elongate tube body 118 also comprises an external body face 126 and an internal body face 124. Opposed receiving faces 128 define a knuckle space 130 for receiving clasp ends 162,163 of an arm rod portion 114 of an insertion instrument 14. FIG. 20 illustrates a cross sectional view along axis G of outer tube housing 116. FIG. 21 is a distal end view and FIG. 22 a proximal end view.

As illustrated in FIG. 21, opposed clearance slots 132 extend through opposed receiving faces 128 and an external body face 126 of an elongate tube body 118 for the passage of surgical cable 100 when insertion instrument 14 is mounted to cable bone screw implant portion 12. Elongate tube body 118 comprises one or more cleaning slots 134 extending between an internal body face 124 and an external body face 126 for eased cleaning of the insertion instrument. An internal chamber 136 of an elongate tube body 118 is sized for sliding engagement with an arm rod portion 114. In some forms, a chamber step 180 transitions between varying diameters of internal body faces 124. External body face 126 comprises a textured surface 138 at a proximal end for enhanced hand gripping by a user during surgery. In one form textured surface 138 is in the form of radial ridges 140 encircling an external body face 126.

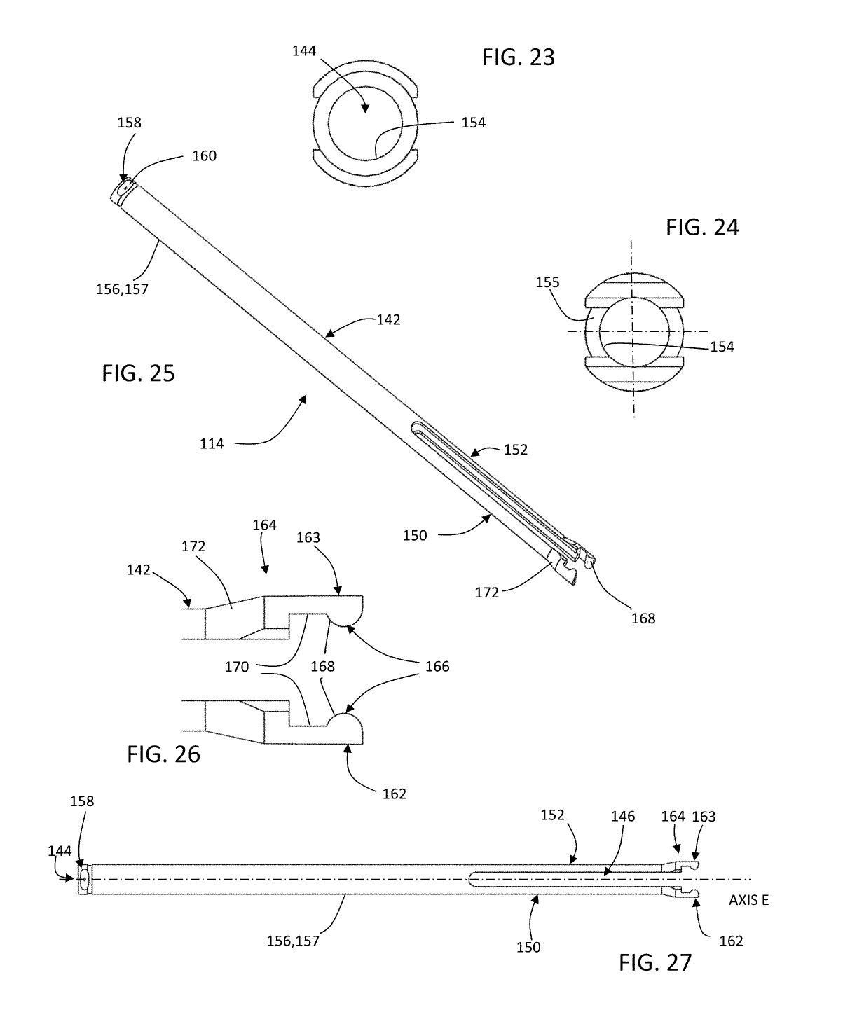

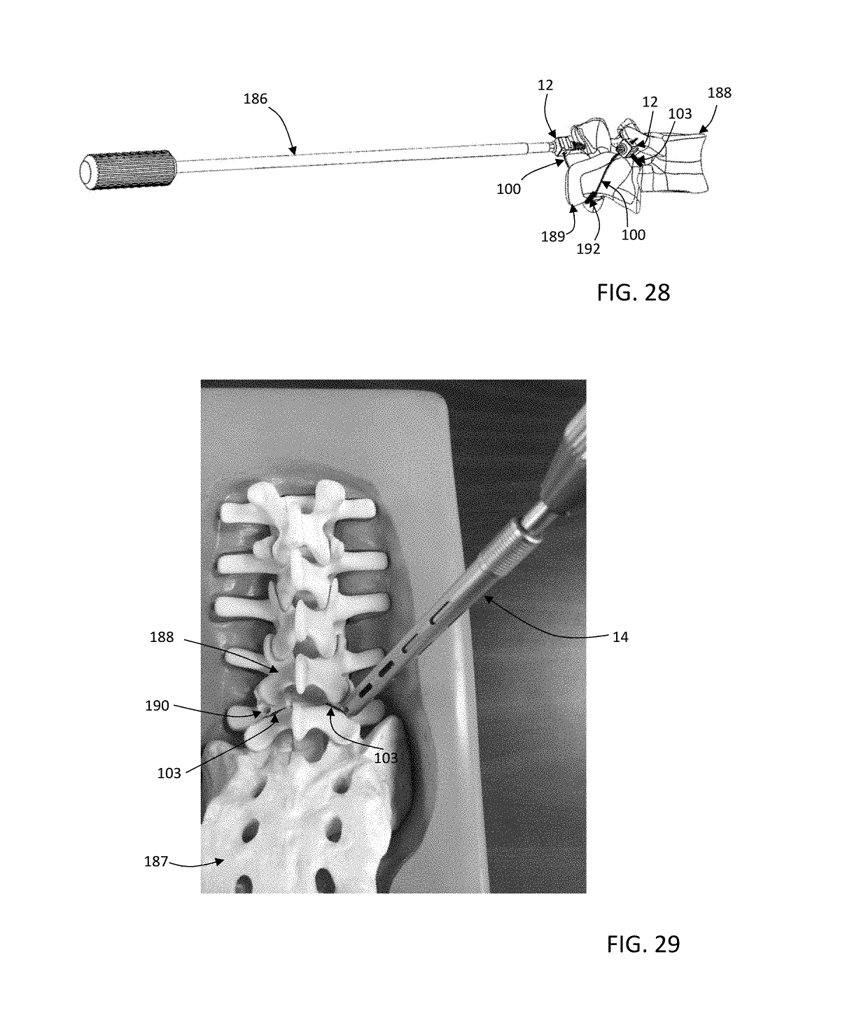

FIGS. 23-27 illustrate an arm rod portion 114 of insertion instrument 14. Arm rod portion 114 comprises an elongate rod body 142 having a driver cannula 144 extending therethrough along a central axis E. Arm rod portion 114 comprises a divider slot 146 splitting a distal end of arm rod portion 114 forming two or more opposed deflector arms 148. This embodiment comprises a first deflector arm 150 and a second deflector arm 152. A driver cannula 144 comprises a cannula face 154 formed thereon and an outer surface 157 encircling the arm rod portion 114 on an outer wall 156. A cup face 155 faces distally.

As illustrated in FIGS. 25 and 27, an arm rod portion 114 comprises a handle lock 158 at a proximal end. Handle lock 158 is in the form of a groove 160, a depression, or a groove and a depression. A deflector arm 150, 152 comprises a clasp end and may be enlarged. In this embodiment, a first deflector arm 150 comprises a first clasp end 162, and a second deflector arm 152 comprises a second clasp end 163. Clasp end 162,163 comprises an interlock 166 for locking engagement with a control mate 34, 36 of an implantable bone screw 16. Here interlock 166 is in the form of an internally facing rounded elongate ridge 168. Interlock 166 includes a head gap portion 170 for extending partially around an enlarged head portion 18 of an implantable bone screw 16. A compression taper 172 extends between a clasp end 162, 163 and an elongate rod body 142.

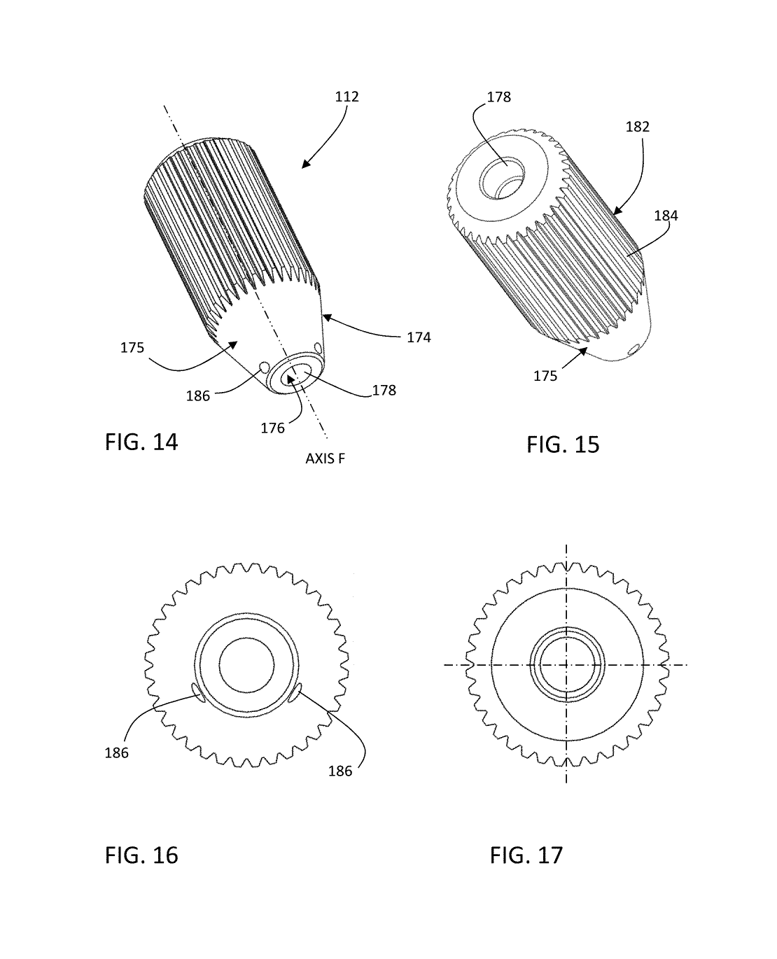

FIGS. 14-17 illustrate one embodiment of a handle portion 112 of an insertion instrument 14. Handle portion 112 comprises an elongate handle body 174 extending along a central axis F and comprises a handle cannula 176 extending along a central axis-F defined by an inner handle surface 178. Handle cannula 176 has a diameter for receiving an outer surface 157 of an arm rod 114 at a distal end. In addition, handle cannula 176 has a diameter sufficient for receiving a locking rod instrument (shown schematically in FIG. 11) extending to a drive pocket 82 of lock screw 66 of implantable bone screw 16. Handle portion 112 comprises a palm surface 180 which is textured 182 for enhanced hand gripping by a user. In this embodiment, a textured palm surface 182 is in the form of a plurality of longitudinal ridges 184 spaced about external handle body surface 175.

Handle portion 112 further comprises lock recess 186 for locking a handle portion 112 to an arm rod portion 114. Lock recess 186 is configured to house a pressed or spring pin (not shown) or a set screw in which case lock recess 186 is threaded for receiving a set screw (not shown).

In this embodiment, locking rod instrument 186 comprises a lock handle 212 at a proximal end and an elongate lock shaft 216 extending from a distal end. A lock face is at the distal most end and comprises in this embodiment a plurality of lobes for exerting torque to a lock screw. Lock handle 212 may be configured to engage a torque limiter to limit input torque.

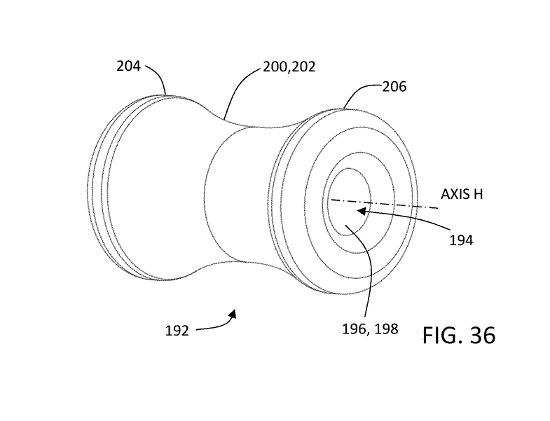

FIG. 36 illustrates one embodiment of a surgical cable grommet 192. It comprises a central grommet aperture 194 sized to pass a surgical cable along axis H. Central grommet aperture 194 is defined by a central wall 196 having central surface 198 thereon. Concave wall 200 with concave surface 202 thereon acts as a seating surface for a corresponding spinous process. Each end of surgical cable grommet 192 is enlarged having a first knuckle 204 at one end and a second knuckle 206 at the other. Surgical cable grommet 192 may be manufactured of a biocompatible metal and alternatively a polymer such as PEEK.

One embodiment of a method of using the disclosed cable bone screw and insertion instrument is disclosed herein. The patient is prepped for surgery by placement of the patient in a prone position over a four-post frame or suitable rolls. The operative area is cleaned and an incision is made over the predetermined site where the cable bone screw will be implanted at the appropriate spinal level. Fluoroscopy or other radiographic techniques may be used during surgery to ensure correct implant placement. Tissue distractors and surgical lighting may be used as needed to reach the bone site. In some cases a guide wire or imaging or both may be used to guide the surgery.

The site of the pars defect or pars fracture is cleaned of soft tissue. Bleeding bone is exposed on both the pedicle and laminar sides preferably keeping facet joints and interspinous ligaments intact. In some cases, a laminotomy is created to decompress the lateral recess and foramen. A small portion of the inferior facet of the vertebrae may be removed to prevent impingement. The exiting nerve root may be exposed to assure it is not compressed. The pedicle is then approached lateral to the facet and superior to the defect.

An awl may be used to initiate the proper entry point into the pedicle. The pathway may be enlarged with a blunt pedicle probe. The position in the pedicle may be checked radiographically to confirm proper placement. The process is repeated for second pedicle. A surgical drill may be optionally used by hand or power to create a pilot hole for the cable bone screw implant portions.

An appropriately sized cable bone screw implant portion 12 is preassembled (preassembled configuration) or assembled in surgery with stress tube 94 housed within insert aperture 40 and lock screw 66 threadably engaged within lock aperture 52. With outer tube housing 116 of insertion instrument 14 retracted proximally toward handle portion 112, clasp ends 162, 163 extend beyond the distal end of outer tube housing 116. Outward spring bias force in deflector arms 150 and 152 cause distancing between opposing interlocks 166 of elongate rod body 142 sufficient to seat enlarged head 18 of implantable bone screw 16 therebetween.

Axis A of implantable bone screw 16 is aligned with axis E of arm rod portion 114. Enlarged head 18 is then raised between clasp ends 162, 163 such that interlocks 166 are aligned with and prepared to occupy corresponding first and second control mates 34 and 36. Grasping outer tube housing 116 and handle portion 112, the user then applies a hand force causing outer tube housing 116 to slide distally causing opposed receiving faces 128 of outer tube housing 116 to squeeze against first and second clasp ends 162, 163 therein engaging and securing implantable bone screw 16 therebetween (loaded configuration).

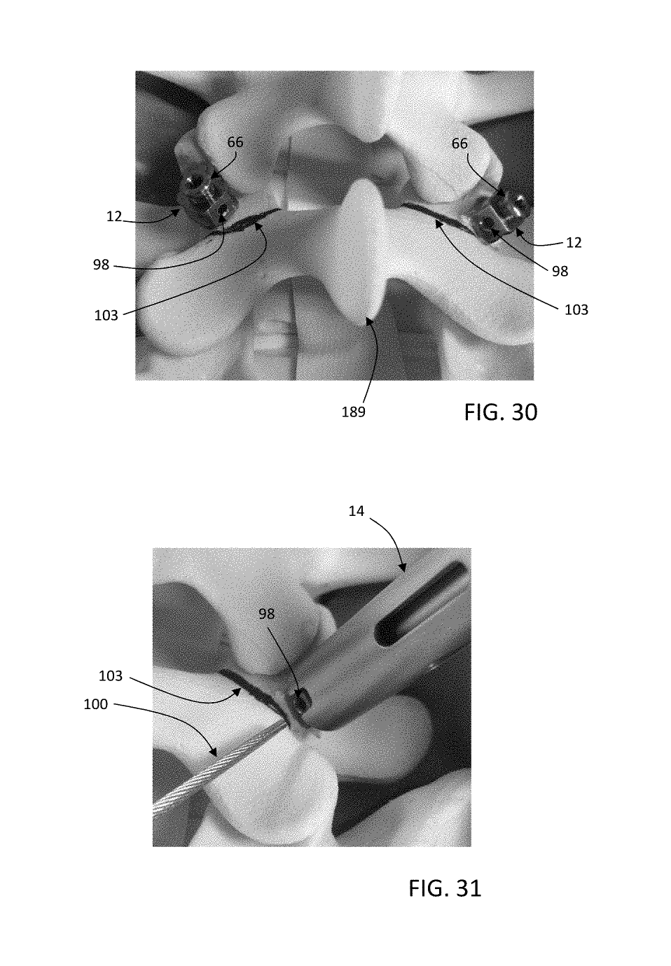

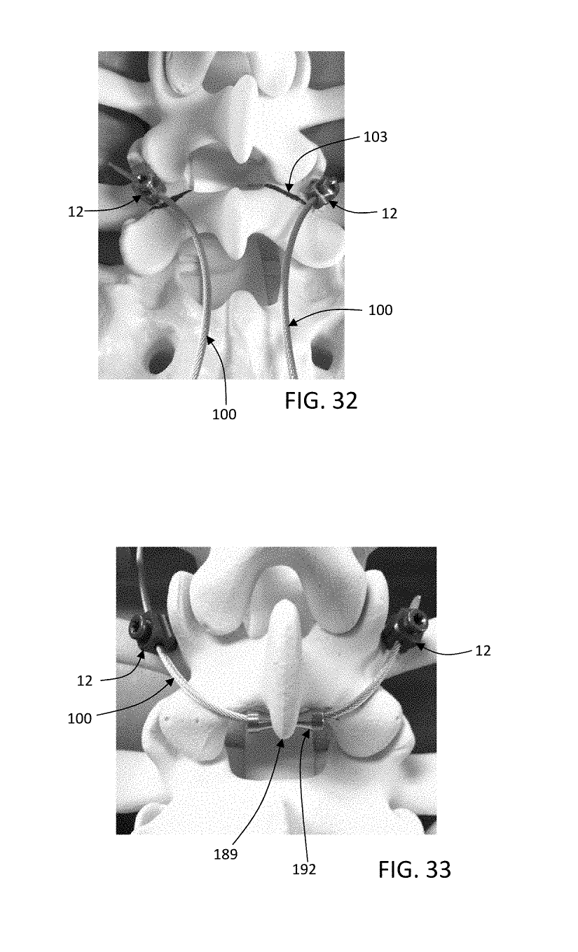

As illustrated in FIG. 29, a cable bone screw implant 12 is then threadably advanced to an appropriate depth in the bone using insertion instrument 14. The direction of the cable bone screw implant 12 is adjusted such that stress aperture 98 is pointed in a direction following a predetermined path of a surgical cable 100. A second cable bone screw implant 12 is then inserted on the contralateral pedicle as illustrated in FIG. 30. The pars defect 103 is prepared which may include insertion of a bone graft into the pars defect per standard surgical techniques such that the graft does not protrude into the spinal canal. A sacrum 187 is illustrated inferiorly. A bored pedicle 190 is illustrated in vertebral body 188.

Using a needle holder or other surgical instrument, surgical cable 100 is threaded through stress aperture 98 as illustrated in FIG. 31. A locking rod instrument 186 extends within the central cannula of insertion instrument 14 and is seated in drive pocket 82. In preferred embodiments this is a hexalobe at distal end of locking rod instrument 186 into hexalobe recess of lock screw 66 mating configuration. Lock screw 66 is advanced by rotation of lock rod instrument 186 causing engagement face 70 of lock screw 66 to crush against second outer tube surface 104 thereby deforming stress tube 94 and collapsing stress aperture 98 such that surgical cable 100 is fixed therein (implanted configuration). A torque limiting handle may be attached to the handle of lock rod instrument 186 to provide measured torque. Alternatively, lock screw 66 advancement can be limited by interference between stop face 78 and proximal head wall surface 32. The cable bone screw implant that is locked first on surgical cable 100 is the“anchoring” cable screw.

A cable grommet 192 is then slid onto the free end of surgical cable 100. The surgical cable 100 is passed around the base of the corresponding spinous process 189 and the cable grommet 192 is aligned beneath the spinous process (FIG. 33). Surgical cable 100 is then passed through the stress tube 94 of the contralateral cable bone screw implant 12 using the shortest path possible.

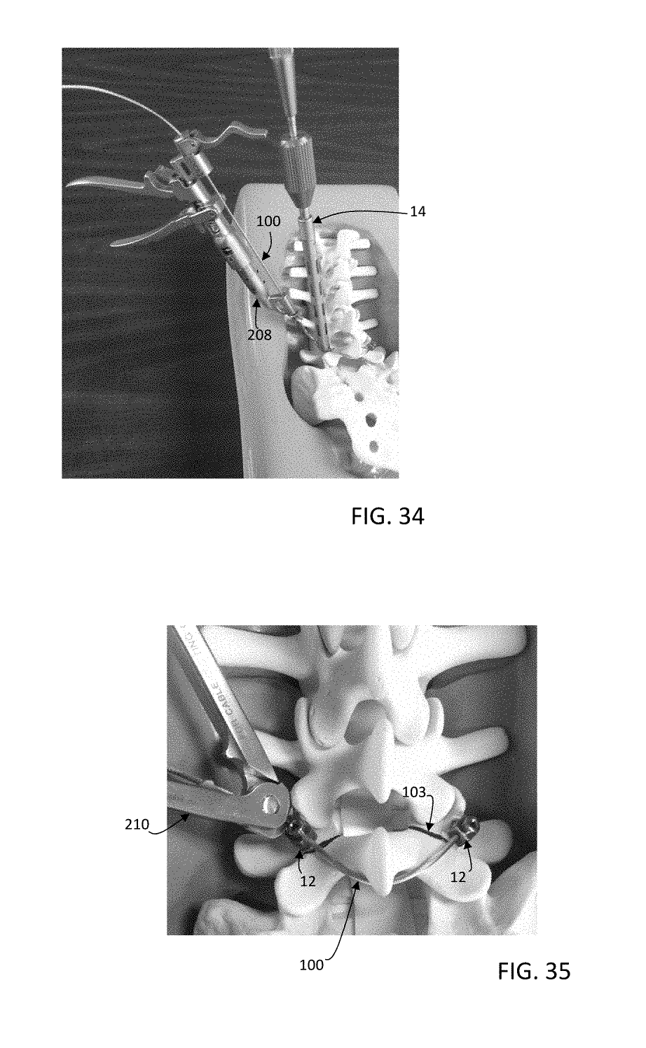

The free end of surgical cable 100 is passed through a cable tensioner 208 per the tensioner manufacturer's surgical technique (FIG. 34). Tension is applied to surgical cable 100 until the desired tension is attained again per the tensioner manufacturer's surgical technique. Once the final tension is achieved, the final lock screw 66 is tightened as described previously. Cable tensioner 208 and lock rod instrument 186 are removed. Lock rod instrument 186 is removed by distracting proximally and outer tube housing 116 is again distracted towards handle portion 112 thus allowing first and second clasp end 162, 163 to spring outwards and release enlarged screw head portion 18. Insertion instrument 14 is distracted proximally from the incision site.

Using a cable cutter 210 (FIG. 35), untensioned portions of surgical cable 100 are cut flush with each cable bone screw implant 12 or as close to the cable bone screw implant as possible with a cable cutter 210. The cable is then removed using surgical forceps or similar instrument. Tension in the cable will create compression between the pedicle, graft, and lamina junction.

If implant removal is needed, the implant is re-exposed. A cable cutter 210 is used to cut the surgical cable 100. The cable is then removed using surgical forceps or similar instrument. An insertion instrument is then engaged to each implanted cable bone screw implant portion and derotated until each screw is removed.

The foregoing invention has been described in accordance with the relevant legal standards, thus the description is exemplary rather than limiting in nature. Variations and modifications to the disclosed embodiment may become apparent to those skilled in the art and fall within the scope of the invention.

What is claimed is:

1. A cable bone screw implant for anchoring a surgical cable to a bone comprising:
an implantable bone screw portion;
said implantable bone screw portion comprising an enlarged head;
said implantable bone screw portion comprising a central axis extending along a shaft portion of said implantable bone screw portion;
said shaft portion comprising bone screw threads formed thereon;
said enlarged head comprising a first radial wall surface and second radial wall surface opposed about said central axis;
said enlarged head comprising a third radial wall surface and fourth radial wall surface opposed about said central axis;
said first radial wall surface and said second radial wall surface generally perpendicular to each other;
said enlarged head comprising a lock aperture extending from a proximal end along said central axis;
said lock aperture defined by a cylindrical lock wall with a lock wall surface formed thereon;
said lock wall surface comprising first lock threads formed thereon;
said enlarged head comprising an insert aperture extending from said third radial wall surface to said fourth radial wall surface along an axis generally perpendicular to said central axis;
said insert aperture defined by a generally cylindrical tunnel wall surface;
a stress tube;
said stress tube comprising an entry wall at one end and an exit wall at an opposing end;
said stress tube comprising a stress aperture extending from said entry wall to said exit wall;
said stress aperture sized for housing a surgical cable therein;
said stress tube comprising a first outer tube surface;
said insert aperture sized for seating at least a portion of said stress tube therein;
a lock screw;
said lock screw comprising a drive pocket on a proximal end of said lock screw;
said lock screw comprising an engagement face on a distal end of said lock screw;
said lock screw comprising an advance face extending between said proximal end and said distal end;
said advance face comprising advance threads thereon;
said lock screw is seated within said lock aperture with intermesh between said first lock threads and said advance threads;
a first control mate disposed on said first radial wall surface for engagement with an inserter instrument;
a second control mate disposed on said second radial wall surface for engagement with an inserter instrument;
and wherein distal advancement of said lock screw causes consequent deformation of said stress tube.

2. The cable bone screw implant of claim 1 wherein at least one of said first control mate and said second control mate are in the form of rounded channel extending generally parallel to said insert aperture.

3. The cable bone screw implant of claim 1 wherein said lock aperture and said insert aperture intersect.

4. The cable bone screw implant of claim 1 wherein said enlarged head comprises a proximal head wall with proximal head wall surface formed thereon at a proximal end of said implantable bone screw portion.

5. The cable bone screw implant of claim 1 wherein said insert aperture through said third radial wall has a diameter that is different than said insert aperture through said fourth radial wall.

6. The cable bone screw implant of claim 1 wherein said stress tube comprises a first outer tube surface extending from one end of said stress tube and a second outer tube surface of a different diameter extending from a second end of said stress tube.

7. The cable bone screw implant of claim 1 wherein said stress tube has at least one tapered entry wall at one end of said stress tube.

8. The cable bone screw implant of claim 1 wherein said lock screw comprises a distally facing stop face for abutting against a proximal head wall of an implantable bone screw thus preventing excessive compression on said stress tube.

9. A cable bone screw implant inserter instrument comprising:
an outer tube housing;
an arm rod portion;
a handle portion;
a locking rod instrument;
said outer tube housing comprising an external body face;
said outer tube housing comprising an internal body face;
said arm rod portion comprising an elongate rod body;
said elongate rod body comprising an outer wall having an outer surface thereon;
said elongate rod body comprising a cannula face extending along an elongate axis of said elongate rod body;
said cannula face defining a driver cannula sized to pass a locking rod instrument for advancing a lock screw of a cable bone screw implant;
wherein a distal end of said elongate rod body is enlarged;
said elongate rod body comprising a divider slot extending from a distal end;
said divider slot defining a first deflector arm and a second deflector arm;
an interlock formed on a medial aspect of said first and second deflector arm for engaging a cable bone screw implant portion;
a head gap portion between said first deflector arm and said second deflector arm sized to seat an enlarged head portion of a cable bone screw implant portion therein;
at least one of said first deflector arm and second deflectors arms comprising a compression taper near a distal end of said arm rod portion for compressing said first deflector arm and second deflector arms medially thus securing a cable bone screw implant portion therein;
said outer tube housing comprising a pair of opposed receiving faces disposed on a distal end of said internal body face for engagement with said first deflector arm and said second deflector arm;
an open clearance slot extending through a distal end of said outer tube housing to provide clearance to a surgical cable;
a cannulated handle portion secured to said arm rod portion at a proximal end of said arm rod portion;
a locking rod instrument;
said locking rod instrument having a lock shaft;
wherein said lock shaft is housed within said driver cannula.

10. A cable bone screw implant inserter instrument of claim 9 wherein said interlock comprises at least one rounded elongate ridge for engagement of a control mate on a cable bone screw implant portion.

11. A cable bone screw implant inserter instrument of claim 9 comprising a cup face positioned at a proximal end of a head gap portion wherein said cup face is generally planar and orientated perpendicular to said elongate axis of said elongate rod body.

12. A cable bone screw implant inserter instrument of claim 9 wherein in a preassembled configuration said first deflector arm and second deflector arm are biased laterally providing sufficient space between said first deflector arm and said second deflector arm to load an enlarged head of a cable bone screw implant portion therein.

13. A cable bone screw implant inserter instrument of claim 9 wherein in a loaded configuration said outer tube housing is slid distally on said arm rod portion causing receiving faces of said outer tube housing to squeeze against said first and second clasp end therein fixing an enlarged head portion of a cable bone screw implant portion therebetween.

14. A cable bone screw implant inserter instrument of claim 9 wherein said outer tube housing comprises a plurality of cleaning slots extending between an internal body face and an external body face.

15. A cable bone screw implant inserter instrument of claim 9 wherein said distal end of said locking rod instrument comprises a driver head of profile and shape for seating within a drive pocket of a lock screw for advancement thereof.

16. A cable bone screw implant inserter instrument of claim 9 wherein said internal body face comprises a translation block extending medially from said internal body face for abutment against a portion of said enlarged clasp end to limit translation of said outer tube housing with respect to said arm rod portion.

17. A cable bone screw implant and insertion instrument assembly comprising:
an implantable bone screw;
said implantable bone screw portion comprising a central axis extending along a shaft portion of said implantable bone screw portion;
said implantable bone screw comprising an enlarged head portion;
said enlarged head portion comprising a lock aperture extending from a proximal end along said central axis;
said enlarged head comprising an insert aperture extending from a radial wall surface along an axis generally perpendicular to said central axis;
a stress tube;
said stress tube comprising a stress aperture extending therethrough;
said stress aperture sized for housing a surgical cable therein;
said stress tube seated in said insert aperture;
a lock screw;
said lock screw comprising a drive pocket on a proximal end of said lock screw;
said lock screw comprising an engagement face on a distal end of said lock screw;
at least one control mate disposed on said enlarged head for engagement with an inserter instrument;
an arm rod portion of an insertion instrument;
said arm rod portion comprising an elongate rod body;
said arm rod portion comprising a centrally placed driver cannula for housing a locking rod instrument;
wherein a distal end of said elongate rod body is enlarged;
said elongate rod body comprising a divider slot extending from a distal end;
said divider slot defining a first deflector arm and a second deflector arm;
an interlock formed on a medial aspect of at least one of said first and second deflector arm for engaging a cable bone screw implant portion;
at least one of said first deflector arm and second deflectors arms comprising a compression taper near a distal end of said arm rod portion for compressing said first deflector arm and second deflector arms medially thus securing a cable bone screw implant portion therein;
an outer tube housing;
said outer tube housing comprising a pair of opposed receiving faces disposed on a distal end of said internal body face for engagement with said first deflector arm and said second deflector arm;
wherein sliding said outer tube housing distally locks said implantable bone screw to said inserter instrument.

18. A cable bone screw implant and insertion instrument assembly of claim 17 further comprising a head gap portion between said first deflector arm and said second deflector arm sized to seat an enlarged head portion of a cable bone screw implant portion therein;

19. A cable bone screw implant and insertion instrument assembly of claim 17 wherein sliding said outer tube housing proximally releases said implantable bone screw from said inserter instrument.

20. A cable bone screw implant and inserter instrument assembly of claim 17 further comprising:
an interlock formed on a medial aspect of said first and second deflector arm for engaging a cable bone screw implant portion;
a control mate disposed on a radial wall surface of said enlarged head for engagement with an inserter instrument;
wherein said interlock comprises at least one rounded elongate ridge for engagement of a control mate on a cable bone screw implant portion.
